(19) United States
(12) Patent Application Publication
(10) Pub. No.: US 2005/0159369 A1
(43) Pub. Date: Jul. 21, 2005

(54) Method of treatment of otitis externa

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: QTM LLC, Weston, CT (US)

(21) Appl. No.: 11/018,312

(22) Filed: Dec. 22, 2004

Related U.S. Application Data
(63) Continuation-in-part of application No. 10/771,330, filed on Feb. 5, 2004.
(60) Provisional application No. 60/505,754, filed on Sep. 26, 2003.
(60) Provisional application No. 60/496,409, filed on Aug. 20, 2003.

(51) Int. Cl. A61K 31/7048; A61K 31/704; A61K 31/496; A61K 31/545

(52) U.S. Cl. 514/28; 514/35; 514/37; 514/39; 514/192; 514/200; 514/253.08; 514/254.07

(57) ABSTRACT

This invention relates to a method of treating otitis externa using a topical combination medication, including one or more antifungal agent such as, for example fluconazole, voriconazole, itraconazole, clotrimazole, amphotericin B, caspofungin, micafungin, terbinafine, naftifine, butenafine, amorolfine, ravuconazole, posaconazole, flucytosine, econazole, enilaconazole, miconazole, oxiconazole, saperconazole, sulconazole, terconazole, tioconazole, nikkomycin Z, anidulafungin (LY303366), nystatin, pimaricin, griseofulvin, ciclopirox, haloprogin, tolnaftate, and undecylenate and one or more antibacterial agent such as neomycin sulfate, polymyxin B sulfate, colistin sulfate, gentamycin, tobramycin, chloramphenicol, Ciprofloxacin, Ofloxacin, a penicillin compound, a cephalosporin compound, a macrolide compound, a fluoroquinolone compound, streptomycin, or kanamycin.

This application is a continuation-in-part of prior co-pending U.S. patent application Ser. No. 10/771,330, filed Feb. 5, 2004, which claims benefit of U.S. Provisional Application Ser. No. 60/496,409, filed Aug. 20, 2003 and U.S. Provisional Application Ser. No. 60/505,754, filed Sep. 26, 2003, the disclosures of all of which are hereby incorporated by reference in their entirety.

### BACKGROUND OF THE INVENTION

1. Technical Field

This invention relates to the field of medical science, and in particular to treatment of otitis externa, whether of fungal, bacterial or mixed etiology, with topical combination medications.

2. Description of the Background Art

Otitis externa is an inflammation of the external auditory canal which can affect people of all ages. This condition is responsible for considerable pain and morbidity. The cause may be bacterial (usually Staphylococcus spp.), fungal, viral (for example herpes zoster oticus), traumatic (usually caused by aggressive ear cleaning), or due to collection (or appearance) of moisture or water. Approximately 10% of otitis externa is primarily of fungal etiology. The remaining 90% is of bacterial or mixed bacterial/fungal origin.

Fungal otitis externa (otomycosis) is a fungal infection of the external auditory canal and generally is caused by (1) Aspergillus niger (80-90% of all cases), (2) Candida albicans and other Candida spp., (3) Actinomyces and (4) Trichophyton. Factors such as hot, humid environments, frequent swimming, chronic bacterial otitis externa, prior treatment of bacterial otitis externa with topical aminoglycosides or other antibacteriologics and suppressed immunity can predispose patients to fungal otitis externa. The number of persons at risk for this infection is increasing due to the liberal and inappropriate use of systemic antibiotics. Patients undergoing bone marrow transplant, solid organ transplant or aggressive chemotherapy for cancer, patients infected with HIV, patients with Type I or Type II diabetes or any immunocompromised individual also may be predisposed to fungal otitis externa.

Many of the same physical factors discussed above can predispose an individual to bacterial otitis externa. The most common precipitants are excessive moisture, which can remove cerumen and increase the pH of the ear canal to provide a better environment for growth of bacteria, and trauma from over-vigorous cleaning of the ears. Bacterial otitis externa usually is dominated by Pseudomonas aeruginosa and Staphylococcus aureus, but often also has a fungal component as well. It is not uncommon for treatment with antibiotics, either systemic or topical, to result in fungal overgrowth. Diagnosis of otitis externa may be confirmed by staining a sample of the exudate with potassium hydroxide (10% KOH) or Gram stain, or by bacterial and/or fungal culture. Culture of the ear exudate, however, is rarely performed unless the infection is particularly severe or resistant or when malignant otitis externa is suspected, whether the otitis is suspected to be of bacterial or fungal origin, or both. In any case, fungal culture can take weeks to grow out sufficiently to identify the fungal species; waiting for fungal culture would delay treatment. Therefore, the usual and customary practice is not to culture the ear before treatment. The treating physician usually does not know the causative organism(s) and treatment is empirical. The compositions currently available for treatment of otitis externa, which are not effective against the common fungal agents in otitis externa, therefore are ineffective in many cases.

Symptoms of otitis externa can include significant ear canal pruritis, pain (particularly with motion of the external ear), otorrhea (usually foul and purulent), conductive hearing loss and cervical lymphadenitis. Whitish-grey, yellow or black ear canal exudate, erythema and swelling of the canal walls, external auditory canal meatus and tympanic membrane, and a distinctive odor are hallmarks of fungal otitis externa. Bacterial otitis externa also can exhibit similar symptoms, such as pain, mucus or bloody discharge from the ear and inflammation. Other symptoms may include hearing loss, tinnitus, fever and others. If otitis externa is severe, it may spread through the skin layers to cartilage and/or bone, and can spread to the face or neck. Necrotizing or malignant otitis externa, a Pseudomonas spp. ostiitis of the temporal bone, may occur, especially in adults with diabetes, as well as in patients who are immunocompromised.

Topical antibiotic (antibacterial) and other preparations are in use to treat otitis of bacterial origin. After cleansing and insertion of a wick if desired, topical agents such as acetic acid, hydrocortisone, Neomycin, polymyxin B, Ofloxacin, Tobramycin, fluoroquinolones and aminoglycosides are applied, or oral antibiotics are administered.

Treatment of otitis externa involving fungal organisms generally entails vigorous ear canal cleaning (ear toilet), irrigation and acidification. Occasionally, surgical debridement of the ear canal is indicated. Current therapy for fungal otitis externa relies on the use of acidifying solutions (for example acetic acid, with or without hydrocortisone) or topical agents designed for treatment of Athlete's Foot (for example clotrimazole (Lotrimin®). Such topical agents are designed for treatment of candidiasis, but generally are not efficacious for many of the organisms known to cause fungal otitis externa and so have proved ineffective. There are no commercially available topical medications indicated for treatment or prophylaxis of fungal or mixed bacterial/fungal otitis externa designed to attack the organisms normally responsible for otitis externa.

Orally active antifungal drugs have been described. See, for example, U.S. Pat. No. 4,404,216. These drugs have been used effectively for invasive fungal infections due to Candida, Aspergillus, and other fungi. Voriconazole has in vitro antifungal activity against a number of species and is considered to be effective in vivo against Candida spp. and Cryptococcus neoformans as well as Aspergillus spp., including fluconazole-resistant Candida species such as C. krusei and C. guilliermondii.

Oral fluconazole (Diflucan®), itraconazole (Sporanox®), voriconazole (Vfend®) and clotrimazole (Mycelex®) have been approved by the FDA for various types of invasive fungal infections. These drugs are synthetic triazole antifungal agents, available as tablets for oral administration. Prescribing information for these drugs list the following indications for usage. Fluconazole: vaginal candidiasis; oropharyngeal and esophageal candidiasis; Candida urinary tract infections, peritonitis, and systemic Candida infections including candidemia, disseminated candidiasis, and pneumoma; and cyptococcal meningitis. Voriconazole: invasive aspergillosis and serious fungal infections caused by Scedosporium apiospermum and Fusarium spp. Itraconazole: blastomycosis, histoplasmosis and aspergillosis in immunocompromised patients and onychomycosis in non-immunocompromised patients. Fluconazole also has been used to decrease the incidence of candidiasis in patients undergoing bone marrow transplantation who receive cytotoxic chemotherapy and/or radiation therapy.

### SUMMARY OF THE INVENTION

An objective of certain embodiments of this invention is to provide a treatment for bacterial and/or fungal otitis externa in a patient, including non-invasive infections, using topical medication. These embodiments of the invention are particularly useful in the situation where a culture of the ear exudate to determine the specific causative organism is not practical before treatment begins.

Accordingly, embodiments of this invention provide a method of treating otitis externa in a patient in need thereof, which comprises topically administering to said patient a combination medication comprising a therapeutically effective amount of an antifungal agent and a therapeutically effective amount of an antibacterial agent. Preferred antifungal agents are fluconazole, voriconazole, itraconazole, caspofungin, clotrimazole and amphotericin B. Other suitable antifungal agents include, but are not limited to micafungin, terbinafine, naftifine, natamycin, butenafine, amorolfine, ravuconazole, posaconazole, flucytosine, econazole, enilaconazole, miconazole, oxiconazole, saperconazole, sulconazole, terconazole, tioconazole, nikkomycin Z, anidulafungin (LY303366), nystatin, pimaricin, griseofulvin, ciclopirox, haloprogin, tolnaftate, and undecylenate. Preferred antibacterial agents are neomycin sulfate, polymyxin B sulfate, colistin sulfate, gentamycin, tobramycin, chloramphenicol, Ciprofloxacin and Ofloxacin. Other suitable antibacterial agents include, but are not limited to polymyxin compounds, penicillins, cephalosporins, macrolides, fluoroquinolones, streptomycin, kanamycin or any antibiotic or bacteriostatic compounds suitable for topical application and effective to kill or inhibit growth of bacterial organisms in the ear.

For topical treatment, the antifungal agent is administered in an amount of about 1 mg/day or about 5,000 mg/day, preferably about 5 mg/day to about 500 mg/day and most preferably about 10 mg/day to about 100 mg/day. The antibacterial agent is administered in an amount of about 0.1 mg/day to about 100 mg/day, preferably about 0.1 mg/day to about 1 mg/day or about 0.1 mg/day to about 0.5 mg/day and most preferably about 0.15 mg/day or about 0.3 mg/day. Using a standard dropper (20 drops per mL) a standard dose of a liquid formulation is about 4 drops, twice a day into each affected ear.

Treatment preferably should be administered for one day or at least 3 days, preferably for about 7 days to about 14 days. Treatment can be for 180 days or longer. The methods are suitable for treating otitis externa that is non-invasive or invasive and which has an etiologic agent which is fungal, bacterial, mixed or unknown.

Additional embodiments of this invention provide a composition for the topical treatment of otitis externa, which comprises an antifungal agent, an antibacterial agent and at least one pharmaceutically acceptable excipient. Preferred antifungal agents are voriconazole, fluconazole, itraconazole, clotrimazole, ravuconazole, posaconazole, miconazole, oxiconazole, saperconazole, sulconazole, terconazole, tioconazole, econazole, enilaconazole, amphotericin B, natamycin, nikkomycin Z, caspofungin, micafungin, anidulafungin, terbinafine, naftifine, butenafine, amorolfine, flucytosine, nystatin, pimaricin, griseofulvin, ciclopirox, haloprogin, tolnaftate, and/or undecylenate. Preferred antibacterial agents are neomycin sulfate, polymyxin B sulfate, colistin sulfate, gentamycin, tobramycin, chloramphenicol, Ciprofloxacin and/or Ofloxacin, but may be any of the following: penicillins, polymyxin compounds, cephalosporins, macrolides, fluoroquinolones, streptomycin, and kanamycin. Most preferred compositions contain at least itraconazole, caspofungin acetate and/or amphotericin B and an antibacterial agent such as neomycin sulfate or a polymyxin.

In yet further embodiments of the invention, the compositions comprise a second antifungal agent, which may be, for example, fluconazole, itraconazole, clotrimazole, ravuconazole, posaconazole, miconazole, oxiconazole, saperconazole, sulconazole, terconazole, tioconazole, econazole, enilaconazole, amphotericin B, natamycin, nikkomycin Z, caspofungin, micafungin, anidulafungin, terbinafine, naftifine, butenafine, amorolfine, flucytosine, nystatin, pimaricin, griseofulvin, ciclopirox, haloprogin, tolnaftate, and/or undecylenate. The compositions also may comprise a second antibacterial agent, which may be, for example, neomycin sulfate, polymyxin B sulfate, colistin sulfate, gentamycin, tobramycin, chloramphenicol, Ciprofloxacin, Ofloxacin, a penicillin, a polymyxin compound a cephalosporin, a macrolide, a fluoroquinolone, streptomycin, or kanamycin.

In further embodiments of the invention, compositions advantageously may further comprise an antiinflammatory agent, for example a topically active steroid such as a corticosteroid, or may further comprise an anesthetic agent, for example lidocaine, or both. Preferably, compositions according to this invention are formulated as an ear drop.

A number of preferred aspects of the invention will be described below.

### DETAILED DESCRIPTION OF THE INVENTION

The antifungal and antibacterial agents preferably are delivered to the effected tissue in a solution or suspension, by medicine dropper, but may be administered using any convenient vehicle, such as powder, cream, ointment, and the like. Formulations such as a solution or powder may be instilled into the ear using an atomizer. Solutions or suspensions generally contain about 1 mg to about 5000 mg antifungal agent per mL of solution or suspension and about 0.1 mg to about 100 mg antibacterial agent per mL of solution or suspension. The solution or suspension may contain about 5 mg to about 2,500 mg antifungal agent per mL and about 0.1 mg to about 1 mg antibacterial agent per mL, and preferably about 10 mg to about 1,000 mg antifungal agent per mL and about 0.1 mg to about 0.5 mg antibacterial agent per mL. Most preferably, the formulation contains about 0.15 or about 0.3 mg per mL.

The solution or suspension can be delivered to the ear canal in amounts of about 0.01 mL to about 5 mL, preferably about 0.1 mL to about 1 mL, or any amount sufficient to fill the canal volume. An ear wick may be used to assist penetration of the agent into the ear canal according to methods known in the art.

Typical treatments with topical formulations according to the invention involve administration of about 0.01 mg/day to about 100 mg/day or preferably about 0.5 mg/day amphotericin B, itraconazole or caspofungin and about 0.01 to about 100 mg or preferably about 0.15 mg/day or about 0.3 mg/day neomycin and/or polymyxin (administered twice daily) for 10 days. The length of treatment preferably is at least 10 days but may extend from 1 day to about 14 days, or until the symptoms are resolved. Preferably, treatment continues for 5 days or more after resolution of symptoms to lessen the chance of recurrence.

Solutions and suspensions for administration of medications to the ear are known in the art and may contain any conventional or pharmaceutically acceptable and suitable excipients. Alternatively, the antifungal and antibacterial agents may be formulated as an ointment, lotion, cream, tincture, paste, aqueous or anhydrous gel, or powder according to traditional methods of formulation known in the pharmaceutical arts and using any conventional and acceptable pharmaceutical excipient or excipients that are known in the art. Topical preparations according to the invention generally are formulated as a liquid and are applied as ear drops, for example using about 4 drops, to the affected ear canal with eardrum held independently. Other methods for administration of other types of topical formulations are known in the art.

Formulations of antifungal and antibacterial agents suitable for use with this invention may contain additional active ingredients in addition to inert pharmaceutical excipients. For example, topical formulations may include hydrocortisone or other corticosteroid agents to assist in reducing inflammation. Such corticosteroids (for example hydrocortisone or dexamethasone) are able to provide synergistically improved effects in topical formulations where inflammation is a problem. Formulations also may contain anesthetic agents such as lidocaine or pontocaine, if desired.

Formulations according to the invention preferably contain, an antifungal agent which is effective against Aspergillus spp., a common cause of fungal otitis externa, or itraconazole, caspofungin or amphotericin B. Other antifungal agents which may form part of the invention include fluconazole, ketoconazole, enilaconazole, econazole, saperconazole, oxiconazole, clotrimazole, micafungin, terbinafine, naftifine, natamycin, butenafine, amorolfine, ravuconazole, posaconazole, flucytosine, miconazole, sulconazole, terconazole, tioconazole, nikkomycin Z, anidulafungin (LY303366), nystatin, pimaricin, griseofulvin, ciclopirox, haloprogin, tolnaftate, and undecylenate.

Treatment methods of the invention may contain any antifungal agent which is effective for the particular causative species of fungus. When the specific causative fungus is not or cannot be identified, or when more than one fungus is present or suspected, itraconazole, caspofungin or amphotericin B preferably is used, alone or in combination with another agent.

Topical medications such as powders and creams which are designed and marketed to treat athlete's foot sometimes have been used in the ear to treat otitis of fungal origin. These products, however, contain clotrimazole or fluconazole, for example, and do not effectively treat most otitis externa. These agents designed to treat athlete's foot may be effective against some Candida species, but are not suitable alone in a general formulation for treatment of otitis externa. Generally, the causative agent(s) of otitis externa are not known and are usually not Candida species. Therefore, these pharmaceutical compositions, which are not effective against Aspergillus niger, the most common causative organism, preferably are not used alone as the antifungal agent in the formulations of the invention here, but may be used as an additional active ingredient in the inventive compositions.

Formulations according to the invention preferably contain, as the antibacterial agent, neomycin sulfate, polymyxin B sulfate or another polymyxin compound, colistin sulfate, gentamycin, tobramycin, chloramphenicol, Ciprofloxacin and/or Ofloxacin. Other suitable agents include one or more of a penicillin, a cephalosporin, a macrolide, a fluoroquinolone, streptomycin, or kanamycin.

Treatment methods of the invention may contain any antibacterial agent which is effective for the particular causative species of bacterial. When the specific causative bacterium(a) is not or cannot be identified, or when more than one bacterial species is present or suspected, Ciprofloxacin preferably is used as the antibacterial agent in the inventive formulation, alone or in combination with another antibacterial agent.

Preferred topical preparations contain one or more additional antifungal compounds such as those listed above and most preferably contain voriconazole as the second antifungal agent. In addition, compounds such as amphotericin B or natamycin are suitable for use as the only or second antifungal agent. Preferred preparations also contain one or more antibacterial compounds such as those listed above and most preferably contain neomycin sulfate, a polymyxin compound Ciprofloxacin or Ofloxacin. Compounds such as polymyxin B sulfate, colistin sulfate, chloramphenicol, gentamycin, tobramycin, or Ofloxacin are suitable for use as the only antibacterial agent. The primary active ingredients, for example itraconazole, caspofungin, amphotericin B or natamycin and neomycin, polymyxin B or Ciprofloxacin, may be combined with a second antifungal and/or antibacterial agent, an anesthetic, an acidifying agent or buffer, a penetration enhancing agent, an anti-inflammatory agent such as a corticosteroid, etc. in a formulation suitable for topical application to the site of infection. Such compositions are effective in the treatment of otitis externa, filling a need in the market, since no effective product indicated for otitis externa is available commercially at this time.

1. A composition for the topical treatment of otitis externa, which comprises an antifungal agent, an antibacterial agent and at least one pharmaceutically acceptable excipient.

2. A composition of claim 1 wherein said antifungal agent is selected from the group consisting of voriconazole, fluconazole, itraconazole, clotrimazole, ravuconazole, posaconazole, miconazole, oxiconazole, saperconazole, sulconazole, terconazole, tioconazole, econazole, enilaconazole, amphotericin B, natamycin, nikkomycin Z, caspofungin, micafungin, anidulafungin, terbinafine, naftifine, butenafine, amorolfine, flucytosine, nystatin, pimaricin, griseofulvin, ciclopirox, haloprogin, tolnaftate, and undecylenate.

3. A composition of claim 1 wherein said antibacterial agent is selected from the group consisting of neomycin sulfate, polymyxin B sulfate, colistin sulfate, gentamycin, tobramycin, chloramphenicol, Ciprofloxacin, Ofloxacin, a penicillin compound, a cephalosporin compound, a macrolide compound, a fluoroquinolone compound, streptomycin, or kanamycin.

4. A composition of claim 1 wherein said antifungal agent is itraconazole.

5. A composition of claim 1 wherein said antifungal agent is caspofungin.

6. A composition of claim 1 wherein said antifungal agent is amphotericin B.

7. A composition of claim 1 wherein said antibacterial agent is neomycin.

8. A composition of claim 1 wherein said antibacterial agent is a polymyxin.

9. A composition of claim 1 which further comprises a second antifungal agent selected from the group consisting of fluconazole, itraconazole, clotrimazole, ravuconazole, posaconazole, miconazole, oxiconazole, saperconazole, sulconazole, terconazole, tioconazole, econazole, enilaconazole, amphotericin B, natamycin, nikkomycin Z, caspofungin, micafungin, anidulafungin, terbinafine, naftifine, butenafine, amorolfine, flucytosine, nystatin, pimaricin, griseofulvin, ciclopirox, haloprogin, tolnaftate, and undecylenate.

10. A composition of claim 1 which further comprises a second antibacterial agent selected from the group consisting of neomycin sulfate, polymyxin B sulfate, colistin sulfate, gentamycin, tobramycin, chloramphenicol, Ciprofloxacin, Ofloxacin, a penicillin compound, a cephalosporin compound, a macrolide compound, a fluoroquinolone compound, streptomycin, or kanamycin.

11. A composition of claim 1 which further comprises an antiinflammatory agent.

12. A composition of claim 11 wherein said anti-inflammatory agent is a corticosteroid.

13. A composition of claim 1 which further comprises an anesthetic agent.

14. A composition of claim 1, which is formulated as a solution, suspension or powder.

15. A method of treating otitis externa in a patient in need thereof, which comprises topically administering to said patient a therapeutically effective amount of a composition of claim 1.

16. A method of claim 15 wherein said antifungal agent is administered in an amount of about 1 to about 5,000 mg/day.

17. A method of claim 15 wherein said antifungal agent is administered in an amount of about 5 to about 500 mg/day.

18. A method of claim 15 wherein said antifungal agent is administered in an amount of about 10 mg/day to about 100 mg/day.

19. A method of claim 15 wherein said antibacterial agent is administered in an amount of about 0.1 mg/day to about 100 mg/day.

20. A method of claim 15 wherein said antibacterial agent is administered in an amount of about 0.1 mg/day to about 1 mg/day.

21. A method of claim 15 wherein said antibacterial agent is administered in an amount of about 0.1 mg/day to about 0.5 mg/day.

22. A method of claim 15 wherein said antibacterial agent is administered in an amount of about 0.15 mg/day.

23. A method of claim 15 wherein said antibacterial agent is administered in an amount of about 0.3 mg/day.

24. A method of claim 15 wherein said composition is administered for at least 3 days.

25. A method of claim 11 wherein said composition is administered for about 7 days to about 14 days.

26. A method of claim 11 wherein said composition is administered for up to 180 days.

27. A method of treating otitis externa in a patient in need thereof, which comprises topically administering to said patient a therapeutically effective amount of a composition which comprises itraconazole, neomycin, a corticosteroid, and at least one pharmaceutical excipient suitable for topical administration.
